(19) United States
(12) Patent Application Publication
(10) Pub. No.: US 2009/0155167 A1
(43) Pub. Date: Jun. 18, 2009

(54) AUTOMATED METHOD

(75) Inventors: Nigel Anthony Powell, Buckinghamshire (GB); Peter Stewart Weisner, Buckinghamshire (GB); Ian Anthony Sargeant, Buckinghamshire (GB); Torgrim Engell, Oslo (NO); John Henrik Johansen, Oslo (NO)

Correspondence Address: GE HEALTHCARE, INC., IP DEPARTMENT, 101 CARNEGIE CENTER, PRINCETON, NJ, 08540-6231, US

(73) Assignee: GE HEALTHCARE LIMITED, Buckinghamshire (GB)

(21) Appl. No.: 12/089,734

(22) PCT Filed: Oct. 9, 2006
(86) PCT No.: PCT/GB06/03745

§ 371 Date: Sep. 9, 2008

(30) Foreign Application Priority Data
Oct. 10, 2005 (GB) 0520527.3

(51) Int. Cl. A61K 51/00; C07F 11/00; G21C 1/00

(52) U.S. Cl. 424/1.61; 556/57; 422/159

(57) ABSTRACT

The present invention provides an automated method for the preparation of ^{99m}Tc radiopharmaceutical compositions, together with disposable cassettes for use in the method. The use of an automated synthesizer apparatus in the preparation of ^{99m}Tc radiopharmaceuticals is also described. Also described is the use of kits for the preparation of ^{99m}Tc radiopharmaceuticals in the method and disposable cassettes of the present invention.

### FIELD OF THE INVENTION

The present invention provides an automated method for the preparation of ^{99m}Tc radiopharmaceutical compositions, together with disposable cassettes for use in the method. The use of an automated synthesizer apparatus in the preparation of ^{99m}Tc radiopharmaceuticals is also described. Also described is the use of kits for the preparation of ^{99m}Tc radiopharmaceuticals in the method and disposable cassettes of the present invention.

### BACKGROUND TO THE INVENTION

Automated methods for the preparation of radiopharmaceuticals which comprise a positron-emitting radioisotope for positron emission tomography (PET) are well-established [D. Alexoff, in “Handbook of Radiopharmaceuticals”, M. J. Welch & C. S. Redvanly (Eds.), pages 283-305 Wiley (2003)].

WO 02/051447 describes an automated synthesizer apparatus for the preparation of radiopharmaceuticals, which incorporates a disposable module containing pre-metered amounts of chemical reagents. The device is said to be particularly useful for the short half-life positron-emitting radioisotopes ¹¹C, ¹³N, ¹⁵O and ¹⁸F.

For ^{99m}Tc radiopharmaceuticals, the conventional wisdom developed over many years is that the user obtains a sterile supply of ^{99m}Tc as ^{99m}Tc-pertechnetate in saline from a ⁹⁹Mo/^{99m}Tc radioisotope generator, and uses that radioactive eluate to reconstitute lyophilised, non-radioactive kits to generate the desired radiopharmaceutical directly in a ready-to-inject form. These steps are typically carried out manually, although efforts have been made to investigate automated radiopharmaceutical dispensing (APD) [Solanki, Hosp. Pharmac., 7(4), 94-98 (2000)]. Automated elution of ⁹⁹Mo/^{99m}Tc radioisotope generators is described in U.S. Pat. No. 4,625,118 and U.S. Pat. No. 5,580,541.

Fisco et al [Lab. Robot. Automat., 6(4), 159-165 (1994)] disclosed the use of a robotic system for the automated kit reconstitution and quality control of the ^{99m}Tc radiopharmaceutical Cardiotec™. Ensing [Dev. Nucl. Med., 22, 49-54 (1992)] reviewed efforts to automate radiopharmaceutical kit preparations in hospital radiopharmacies, including automated reconstitution of non-radioactive kits.

Prior art approaches have therefore focused on either the automated reconstitution of either ^{99m}Tc generators or ^{99m}Tc kits, always with the conventional kit as the basis for the chemistry involved. This has the drawback that, if multiple doses are required on a regular basis, the only option is to process large numbers of kits, with each kit requiring a separate QC check on radiochemical purity (RCP). This means that the same process steps may need to be replicated many times over, which is inefficient, and that the volume and number of containers and apparatus made radioactive as a result of the operations is relatively high. Whilst automation is recognised as having the potential to reduce operator radiation dose, prior art automated processes have also been reported to be much slower than the manual counterpart, which makes them less attractive [Solanki, Hosp. Pharmac., 7(4), 94-98 (2000)]. There is therefore a need for an automated approach which is fast, more flexible, less constrained by existing kit chemistry, and which can generate larger batch sizes in a more efficient manner.

### THE PRESENT INVENTION

The present invention provides an automated method for the preparation of ^{99m}Tc radiopharmaceutical compositions, together with disposable cassettes for use in the method. The method is particularly suitable for use in conjunction with “automated synthesizer” apparatus which are commercially available, but currently used primarily for the preparation of short-lived PET radiopharmaceuticals. The method is particularly useful where large numbers of unit patient doses are required on a regular basis, such as in a radiopharmacy serving either multiple hospitals or a single large hospital. This permits a single determination of RCP.

The present invention also permits the preparation of sterile ^{99m}Tc radiopharmaceuticals which are not amenable to preparation via the conventional kit approach, due to eg. the need to use non-aqueous solvents or where undesirable non-biocompatible impurities cannot easily be removed within the ambit of the kit approach.

The method can be readily adapted to use ⁹⁹Mo-molybdate in solution as the source of ^{99m}Tc-pertechnetate, as opposed to the conventional ⁹⁹Mo/^{99m}Tc generator. The use of a radioactive starting material in solution makes automation of the processes involved more straightforward, and thus avoids the complexities of the prior art needed to automate generator elution.

The cassettes of the present invention contain the non-radioactive chemicals necessary for a given ^{99m}Tc radiopharmaceutical preparation, and may optionally also include the necessary radioactive precursor chemicals. These cassettes make the present method more flexible than prior art approaches. Use of the cassettes in the preparation of ^{99m}Tc radiopharmaceuticals is also described.

The present invention also provides the use of automated synthesizer apparatus for ^{99m}Tc radiopharmaceutical preparation, plus the use of sterile, non-radioactive kits in the present claimed method of preparation.

### DETAILED DESCRIPTION OF THE INVENTION

In a first aspect, the present invention provides an automated method for the preparation of a sterile, ^{99m}Tc radiopharmaceutical composition which comprises a ^{99m}Tc metal complex in a biocompatible carrier medium, wherein said method comprises:
(i) provision of a precursor which comprises a solution of ^{99m}Tc-pertechnetate;
(ii) provision of a supply of a non-radioactive ligand, wherein said ligand forms a metal complex with ^{99m}Tc;
(iii) provision of a supply of a reductant capable of reducing technetium from the Tc(VII) oxidation state to a lower technetium oxidation state;
(iv) complexation of the ligand with ^{99m}Tc by microprocessor-controlled transfer of separate aliquots of said precursor and ligand to a reaction vessel and mixing therein, with optional heating, and optionally in the presence of an amount of said reductant effective to reduce said aliquot of ^{99m}Tc-pertechnetate precursor;
(v) when the ^{99m}Tc complex product from step (iv) is already in a biocompatible carrier medium it is used directly in step (vi), otherwise the product of step (iv) is either dissolved in a biocompatible carrier medium or the solvent used in step (iv) is removed and the residue re-dissolved in a biocompatible carrier medium;
(vi) optionally carrying out one or more of the following additional processes: purification; pH adjustment; solvent removal and re-dissolution in a biocompatible solvent to give the desired ^{99m}Tc radiopharmaceutical composition;
(vii) either maintaining sterility during steps (i) to (vi) so that the ^{99m}Tc metal complex from step (vi) is already sterile, or subjecting the ^{99m}Tc metal complex from step (vi) to either terminal sterilisation or sterile filtration to give the desired ^{99m}Tc-radiopharmaceutical.

The “biocompatible carrier medium” is a fluid, especially a liquid, in which the ^{99m}Tc metal complex is suspended or dissolved, such that the composition is physiologically tolerable, ie. can be administered to the mammalian body without toxicity or undue discomfort. The biocompatible carrier medium is suitably an injectable carrier liquid such as sterile, pyrogen-free water for injection; an aqueous solution such as saline (which may advantageously be balanced so that the final product for injection is either isotonic or not hypotonic); an aqueous solution of one or more tonicity-adjusting substances (eg. salts of plasma cations with biocompatible counterions), sugars (e.g. glucose or sucrose), sugar alcohols (eg. sorbitol or mannitol), glycols (eg. glycerol), or other non-ionic polyol materials (eg. polyethyleneglycols, propylene glycols and the like). The biocompatible carrier medium may also comprise biocompatible organic solvents such as ethanol. Such organic solvents are useful to solubilise more lipophilic compounds or formulations. Preferably the biocompatible carrier medium is pyrogen-free water for injection, isotonic saline or an aqueous ethanol solution. As indicated above, the pH of the biocompatible carrier medium for intravenous injection is suitably in the range 4.0 to 10.5.

The term “microprocessor-controlled” has its conventional meaning. Thus, the term “microprocessor” as used herein, refers to a computer processor contained on an integrated circuit chip, such a processor may also include memory and associated circuits. The microprocessor is designed to perform arithmetic and logic operations using logic circuitry that responds to and processes the basic instructions that drive a computer. The microprocessor may also include programmed instructions to execute or control selected functions, computational methods, switching, etc. Microprocessors and associated devices are commercially available from a number of sources, including, but not limited to: Cypress Semiconductor Corporation, San Jose, Calif.; IBM Corporation; Applied Microsystems Corporation, Redmond, Wash., USA; Intel Corporation and National Semiconductor, Santa Clara, Calif. With regard to the present invention, the microprocessor provides a programmable series of reproducible steps involving eg. transfer of chemicals, heating, filtration etc. The microprocessor of the present invention also preferably records batch production data (eg. reagents used, reaction conditions, radioactive materials etc). This recorded data is useful to demonstrate GMP compliance for radiopharmaceutical manufacture. The microprocessor is also preferably linked to a barcode reader to permit facile selection of reaction conditions for a given production run, as described below.

The term “oxidation state” has its conventional meaning in inorganic chemistry. By the term “lower technetium oxidation state” is meant Tc(—I) to Tc(VI). Preferred oxidation states for the ligand metal complex with ^{99m}Tc are in the range Tc(0) to Tc(V), and are most preferably chosen from Tc(I), Tc(III) and Tc(V). Technetium complexes of ligands having an oxidation state Tc(VII) are, however, known. For such complexes a reductant may not be necessary. For technetium complexes of oxidation state Tc(—I) to Tc(VI), the reductant is expected to be an essential feature of the method of the present invention.

The oxidation state of the technetium in ^{99m}Tc-pertechnetate is Tc(VII). The “reductant” of the present invention is suitable for reduction of Tc(VII) pertechnetate to lower oxidation states of technetium, ie. the oxidation state of technetium in the metal complex of ^{99m}Tc with the ligand. Suitable such reductants are known in the art [Clarke, Coord Chem. Rev., 78, 253-331 (1987) and references therein]. It is also envisaged that the reduction could be carried out using an electrolytic cell, which could form an additional feature of the cassette of the present invention. Such electrolytic cells have the advantage of providing controlled reduction conditions, with the need to add chemical reductants. The reductant of the present invention does not have to be biocompatible, since the flexibility of the method means that non-biologically compatible reductants can subsequently be removed. Biocompatible reductants are, however, preferred. By the term “biocompatible reductant” is meant a reducing agent suitable for reduction of Tc(VII) pertechnetate to lower oxidation states of technetium, which is non-toxic at the required dosage and hence suitable for administration to the mammalian body, especially the human body. Suitable such reductants include: sodium dithionite, sodium bisulphite, ascorbic acid, formamidine sulphinic acid, stannous ion, Fe(II) or Cu(I). The biocompatible reductant is preferably a stannous salt such as stannous chloride or stannous tartrate.

The reductant of the present invention may be supplied in solid (eg. lyophilised) or solution form. When used in solid form, a known amount of reductant is suitably provided in a vial or container, and dissolved in a suitable solvent prior to use, as part of the automated method. When used in solution form, this has the advantage that the reductant concentration is known and hence the microprocessor-controlled delivery of the right amount of reductant simplifies to the delivery of a specific volume or aliquot of reductant solution. The reductant solution is preferably in a biocompatible carrier medium, as defined above. Sterile solutions of stannous in biocompatible carrier media are expected to be sufficiently stable in the absence of air in a suitable container to have a useful shelf-life for use in the cassette of the present invention.

The term “ligand” as used herein has its conventional meaning in inorganic chemistry, ie. a compound which forms a complex with a metal, in this instance technetium. By the term “metal complex” is meant a coordination complex of the metal ion with one or more ligands. It is strongly preferred that the technetium metal complex is “resistant to transchelation”, ie. does not readily undergo ligand exchange with other potentially competing ligands for the ^{99m}Tc coordination sites. Potentially competing ligands include other excipients in the preparation in vitro (eg. radioprotectants, antimicrobial preservatives or sterilising agents such as alcohols used in the preparation), or endogenous compounds in vivo (eg. glutathione, transferrin or plasma proteins). Suitable ligands for use in the present invention which form technetium complexes resistant to transchelation include: chelating agents, where 2-6, preferably 2-4, metal donor atoms are arranged such that chelate rings result (by having a non-coordinating backbone of either carbon atoms or non-coordinating heteroatoms linking the metal donor atoms), preferably 5- or 6-membered chelate rings; or monodentate ligands which comprise donor atoms which bind strongly to the technetium, such as carbon monoxide (CO), isonitriles, phosphines, thiols or diazenides.

Examples of donor atom types which bind well to technetium as part of chelating agents are: amines, thiols, amides, oximes and phosphines. Phosphines form such strong metal complexes that even monodentate or bidentate phosphines form suitable technetium complexes. The linear geometry of isonitriles and diazenides is such that they do not lend themselves readily to incorporation into chelating agents, and are hence typically used as monodentate ligands. Examples of suitable isonitriles include simple alkyl isonitriles such as tert-butylisonitrile, and ether-substituted isonitriles such as mibi (i.e. 1-isocyano-2-methoxy-2-methylpropane). Examples of suitable phosphines include Tetrofosmin, and monodentate phosphines such as tris(3-methoxypropyl)phosphine. Examples of suitable diazenides include the HYNIC series of ligands i.e. hydrazine-substituted pyridines or nicotinamides.

Examples of suitable chelating agents for technetium which form metal complexes resistant to transchelation include, but are not limited to:

### (i) Diaminedioximes of Formula:

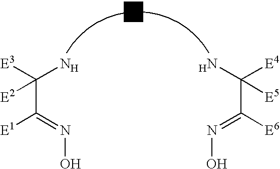

where E¹-E⁶ are each independently an R′ group;
each R′ is H or C₁₋₁₀ alkyl, C₃₋₁₀ alkylaryl, C₂₋₁₀ alkoxyalkyl, C₁₋₁₀ hydroxyalkyl, C₁₋₁₀ fluoroalkyl, C₂₋₁₀ carboxyalkyl or C₁₋₁₀ aminoalkyl, or two or more R′ groups together with the atoms to which they are attached form a carbocyclic, heterocyclic, saturated or unsaturated ring;
and Q is a bridging group of formula -(J)_{f}-;
where f is 3, 4 or 5 and each J is independently —O—, —NR′— or —C(R′)₂— provided that -(J)_{f}- may contain a maximum of one J group which is —O— or —NR′—.

Preferred Q groups are as follows:

Q=—(CH₂)(CHR′)(CH₂)— ie. propyleneamine oxime or PnAO derivatives;
Q=—(CH₂)₂(CHR′)(CH₂)₂— ie. pentyleneamine oxime or PentAO derivatives;

### Q=—(CH₂)₂NR′(CH₂)₂—.

E¹ to E⁶ are preferably chosen from: C₁₋₃ alkyl, alkylaryl alkoxyalkyl, hydroxyalkyl, fluoroalkyl, carboxyalkyl or aminoalkyl. Most preferably, each E¹ to E⁶ group is CH₃.

Q is preferably —(CH₂)(CHR′)(CH₂)—, —(CH₂)₂(CHR′)(CH₂)₂— or —(CH₂)₂NR′(CH₂)₂—, most preferably —(CH₂)₂(CHR′)(CH₂)₂—. An especially preferred bifunctional diaminedioxime chelator has the Formula:

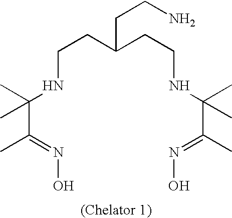

wherein the bridgehead primary amine group can be conjugated to a variety of biological targeting molecules, as is known in the art.
(ii) N₃S ligands having a thioltriamide donor set such as MAG₃ (mercaptoacetyltriglycine) and related ligands; or having a diamidepyridinethiol donor set such as Pica;
(iii) N₂S₂ ligands having a diaminedithiol donor set such as BAT or ECD (i.e. ethylcysteinate dimer), or an amideaminedithiol donor set such as MAMA;
(iv) N₄ ligands which are open chain or macrocyclic ligands having a tetramine, amidetriamine or diamidediamine donor set, such as cyclam, monoxocyclam or dioxocyclam;
(v) N₂O₂ ligands having a diaminediphenol donor set.

The above described ligands and their complexation with technetium are described more fully by Jurisson et al [Chem. Rev., 99, 2205-2218 (1999)].

Preferred ligands of the present invention are chosen from: phosphines; isonitriles and chelating agents which are tetradentate. Preferred such tetradentate chelating agents include: diaminedioximes; N₄ chelating agents having a tetramine, amidetriamine or diamidediamine donor set; N₃S chelating agents having a thioltriamide donor or diamidepyridinethiol donor set; or N₂S₂ chelating agents having a diaminedithiol donor set such as BAT or an amideaminedithiol donor set such as MAMA. Preferred such ligands include: the N₄, N₃S and N₂S₂ chelating agents described above, most preferably N₄ tetramine, diaminedioxime and N₂S₂ diaminedithiol or diamidedithiol chelating agents, especially the N₂S₂ diaminedithiol chelator known as BAT, or variants thereof without the gem-dimethyl groups:

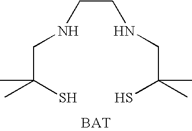

The ligand of the present invention may optionally be conjugated to biological targeting molecules as is known in the art [Banerjee et al, Semin. Nucl. Med., 31(4), 260-277 (2001)].

The method of the present invention may be carried out under aseptic manufacture conditions to give the desired sterile, non-pyrogenic radiopharmaceutical product, as described in eg. US Pharmacopoeia Guidelines. The initial steps (i) to (vi) may also be carried out under non-sterile conditions, followed by sterilisation by either sterile filtration or terminal sterilisation using e.g. gamma-irradiation, autoclaving, dry heat or chemical treatment (e.g. with ethylene oxide). Preferably, sterility is maintained during steps (i) to (vi) such that no additional terminal sterilisation step is necessary.

The precursor, ligand, reducing agent and reaction vessel are each supplied in suitable vials or vessels which comprise a sealed container which permits maintenance of sterile integrity and/or radioactive safety, plus optionally an inert headspace gas (eg. nitrogen or argon), whilst permitting addition and withdrawal of solutions by syringe or cannula. A preferred such container is a septum-sealed vial, wherein the gas-tight closure is crimped on with an overseal (typically of aluminium). The closure is suitable for single or multiple puncturing with a hypodermic needle (e.g. a crimped-on septum seal closure) whilst maintaining sterile integrity. Such containers have the additional advantage that the closure can withstand vacuum if desired (eg. to change the headspace gas or degas solutions), and withstand pressure changes such as reductions in pressure without permitting ingress of external atmospheric gases, such as oxygen or water vapour.

The ^{99m}Tc radiopharmaceutical composition products of the method of the present invention are suitably supplied in a sealed container as described above, which may contain single or multiple patient doses. Single patient doses or “unit doses” can thus be withdrawn into clinical grade syringes at various time intervals during the viable lifetime of the preparation to suit the clinical situation. Preferred multiple dose containers comprise a single bulk vial (e.g. of 10 to 30 cm³ volume) which contains sufficient radioactivity for multiple patient doses. Unit dose syringes are designed to be used with a single human patient only, and are therefore preferably disposable and suitable for human injection. The filled unit dose syringes may optionally be provided with a syringe shield to protect the operator from radioactive dose. Suitable such radiopharmaceutical syringe shields are known in the art and preferably comprise either lead or tungsten.

The term “kit” has its conventional meaning in ^{99m}Tc radiopharmaceutical chemistry and refers to a non-radioactive formulation, containing the necessary reactants in a convenient chemical form so that the preparation of the radiopharmaceutical can be carried out in a straightforward manner. Such kits are designed to give sterile radiopharmaceutical products suitable for human administration, e.g. via direct injection into the bloodstream. The kit is preferably lyophilised and is designed to be reconstituted with sterile ^{99m}Tc-pertechnetate (TcO₄⁻) to give a solution suitable for human administration without further manipulation. Suitable kits comprise a sealed container, as described above, containing the ligand in either free base or acid salt form. Preferably, the kit further comprises a “biocompatible reductant” as defined above, also in sterile, lyophilised form. Alternatively, the kit may optionally contain a non-radioactive metal complex of the ligand which, upon addition of the ^{99m}Tc, undergoes transmetallation (i.e. metal exchange) giving the desired ^{99m}Tc metal complex product. An example of this is the copper isonitrile complex used in kits for the preparation of ^{99m}Tc isonitrile complexes.

The non-radioactive kits may optionally further comprise additional components such as a transchelator, radioprotectant, antimicrobial preservative, pH-adjusting agent or filler. The “transchelator” is a compound which reacts rapidly to form a weak complex with the radiometal, then is displaced by the ligand. For technetium, this minimises the risk of formation of reduced hydrolysed technetium (RHT) due to rapid reduction of pertechnetate competing with technetium complexation. Suitable such transchelators are salts of a weak organic acid, ie. an organic acid having a pKa in the range 3 to 7, with a biocompatible cation. Suitable such weak organic acids are acetic acid, citric acid, tartaric acid, gluconic acid, glucoheptonic acid, benzoic acid, phenols or phosphonic acids. Hence, suitable salts are acetates, citrates, tartrates, gluconates, glucoheptonates, benzoates, phenolates or phosphonates. Preferred such salts are tartrates, gluconates, glucoheptonates, benzoates, or phosphonates, most preferably phosphonates, most especially diphosphonates. By the term “biocompatible cation” is meant a positively charged counterion which forms a salt with an ionised, negatively charged group, where said positively charged counterion is also non-toxic and hence suitable for administration to the mammalian body, especially the human body. Examples of suitable biocompatible cations include: the alkali metals sodium or potassium; the alkaline earth metals calcium and magnesium; and the ammonium ion. Preferred biocompatible cations are sodium and potassium, most preferably sodium. A preferred such transchelator is a salt of MDP, ie. methylenediphosphonic acid, with a biocompatible cation.

By the term “radioprotectant” is meant a compound which inhibits degradation reactions, such as redox processes, by trapping highly-reactive free radicals, such as oxygen-containing free radicals arising from the radiolysis of water. The radioprotectants of the present invention are suitably chosen from: ascorbic acid, para-aminobenzoic acid (ie. 4-aminobenzoic acid), gentisic acid (ie. 2,5-dihydroxybenzoic acid) and salts thereof with a biocompatible cation as described above.

By the term “antimicrobial preservative” is meant an agent which inhibits the growth of potentially harmful micro-organisms such as bacteria, yeasts or moulds. The antimicrobial preservative may also exhibit some bactericidal properties, depending on the dose. The main role of the antimicrobial preservative(s) of the present invention is to inhibit the growth of any such micro-organism in the radiopharmaceutical composition post-reconstitution, ie. in the radioactive diagnostic product itself. The antimicrobial preservative may, however, also optionally be used to inhibit the growth of potentially harmful micro-organisms in one or more components of the non-radioactive kit of the present invention prior to reconstitution. Suitable antimicrobial preservative(s) include: the parabens, ie. methyl, ethyl, propyl or butyl paraben or mixtures thereof; benzyl alcohol; phenol; cresol; cetrimide and thiomersal. Preferred antimicrobial preservative(s) are the parabens.

The term “pH-adjusting agent” means a compound or mixture of compounds useful to ensure that the pH of the reconstituted kit is within acceptable limits (approximately pH 4.0 to 10.5) for human or mammalian administration. Suitable such pH-adjusting agents include pharmaceutically acceptable buffers, such as tricine, phosphate or TRIS [ie. tris(hydroxymethyl)aminomethane], and pharmaceutically acceptable bases such as sodium carbonate, sodium bicarbonate or mixtures thereof. The pH adjusting agent may optionally be provided in a separate vial or container, so that the user of the kit can adjust the pH as part of a multi-step procedure.

By the term “filler” is meant a pharmaceutically acceptable bulking agent which may facilitate material handling during production and lyophilisation. Suitable fillers include inorganic salts such as sodium chloride, and water soluble sugars or sugar alcohols such as sucrose, maltose, mannitol or trehalose.

Preferred kits for use in the present invention comprise a ligand chosen from a: phosphine, isonitrile, diaminedioxime, bis(aminothiol) or mercaptoacetyltriglycine (MAG3). Especially preferred kits for use in the present invention are those which comprise the ligands: tetrofosmin, mibi, exametazime, bicisate (ECD), ECD diacid or mercaptoacetyltriglycine (MAG3), MDP and Chelator 1 (as defined above).

When step (vi) of the present invention includes a purification step, this could include one or more of the following:
(i) filtration to remove unwanted insoluble matter or particulates;
(ii) chromatography;
(iii) cartridge purification e.g. Sep-pak.
   The chromatography may involve conventional normal phase or reverse phase methodology, or ion exchange methods. In some instances the desired product is essentially immobilised at the top of a column matrix because of much higher affinity for the stationary phase compared to the mobile phase. The impurities can thus be eluted in a mobile phase to which they have higher affinity than the stationary phase to a suitably shielded waste container. After washing, the purified product can subsequently simply be eluted using an alternative eluent system to which the product exhibits higher affinity than the stationary phase. Any such chromatography is preferably carried out using disposable columns, so that there is no risk that subsequent preparations are contaminated with material from previous preparations. Such disposable columns are commercially available.

When step (vi) of the present invention includes a pH adjustment step, this can be carried out using a pH-adjusting agent as described above.

When step (v) or step (vi) of the present invention includes solvent removal and re-dissolution steps, the solvent can be removed by various techniques:
(i) chromatography;
(ii) application of reduced pressure or vacuum;
(iv) evaporation due to heating or bubbling of gas through or over the solution;
(v) Cartridge purification e.g. Sep-pak.

The chromatography technique applies immobilisation as described above, and is a preferred method. Such solvent removal techniques are important because they permit the preparation of ^{99m}Tc complexes by reaction in organic solvents, but the final radiopharmaceutical is still supplied in a biocompatible carrier medium. This is particularly useful for ligands or intermediates which are either poorly soluble in aqueous media or perhaps susceptible to hydrolysis in free ligand form, but stable as the Tc-ligand metal complex. Examples of the former are arene- and cyclopentadienyl-containing ligands. Examples of the latter are imine or Schiff base ligands, some of which are also poorly soluble in water. Hence, when the ligand is poorly soluble or susceptible to hydrolysis in aqueous media, the solvent used for the solution of step (ii) is preferably an organic solvent, most preferably a water-miscible organic solvent such as acetonitrile, ethanol, dimethylformamide, dimethylsulfoxide or acetone. Preferred such solvents are acetonitrile, ethanol and dimethylsulfoxide.

A further important example of a class of complexes which have interesting biological properties but which are not amenable to conventional kit technology are the technetium tricarbonyl complexes, ie. complexes of the type ^{99m}Tc(CO)₃(ligand). Whilst a kit for the preparation of [^{99m}Tc(CO)₃(H₂O)₃]⁺ has been described, it is not for human use (ie. for in vitro research purposes only) [Schibli, Eur. J. Nucl. Med., 29(11), 1529-1542 (2002)]. The method of the present invention is particularly useful for the preparation of such ^{99m}Tc(CO)₃(ligand) complexes.

The purification method may also involve removal of excess non-radioactive ligand from the technetium-ligand complex. This is particularly important when the uncomplexed ligand is also biologically active (eg. a peptide with affinity for a given receptor in vivo), since that removes any possibility of the uncomplexed ligand competing with the ^{99m}Tc-ligand metal complex for the biological target site of interest in vivo. Excess ligand can be removed either during the purification steps described above or by using a solid phase approach. Chromatography is the preferred method of separation. In cases where the solubility of the ^{99m}Tc complex is very different from the uncomplexed material in a given solvent, precipitation of the free ligand and filtration are also possible. When chromatographic methods are used, a disposal cartridge system is preferred, but a preparative HPLC system is also suitable.

The precursor solution of ^{99m}Tc-pertechnetate is preferably sterile, and supplied by elution of a suitable ^{99m}Tc radioisotope generator. The elution may have already been carried out as a separate exercise, or the elution may optionally be arranged such that as an additional feature, the present process further includes the automated elution of the ^{99m}Tc generator.

In a preferred embodiment, the present method further comprises a sterile reservoir solution of ⁹⁹Mo-molybdate in a suitable solvent, wherein the ^{99m}Tc-pertechnetate precursor of step (i) is provided by in situ radioactive decay of said ⁹⁹Mo to ^{99m}Tc, and said ^{99m}Tc-pertechnetate is separated from the ⁹⁹Mo-molybdate as part of the same automated process under microprocessor control. Such separation methods are known in the art and include: chromatography, sublimation and solvent extraction. A preferred such method is chromatography.

Allowance must be made for a suitable time to elapse to permit the radioactive decay of ⁹⁹Mo to generate a suitable amount of ^{99m}Tc (⁹⁹Mo half-life is 66 hours). Preferably the solvent for the ⁹⁹Mo-molybdate comprises a biocompatible carrier medium, as defined above, most preferably saline. This has the advantage that elution of a conventional ^{99m}Tc generator is not needed for use in this preferred embodiment of the present method. Instead, the customer is supplied with sterile, pyrogen-free supply of ⁹⁹Mo-molybdate which may comprise additional chemical components such as oxidising agents, as is described in the sixth embodiment below.

Aliquots from the ⁹⁹Mo-molybdate reservoir are dispensed under microprocessor control onto a chromatography column suitable for the separation of pertechnetate from molybdate. Suitable materials for the separation column which give highly efficient separation are known in the art and include alumina and zirconia, and are reviewed by Molinski [Int. J. Appl. Rad. Isot., 33, 811-819 (1982). The separation column may be designed for single-use or for multiple use, ie. single elution or multiple elution with each elution giving ^{99m}Tc-pertechnetate for use in the method of the present invention. For multiple use columns, the ⁹⁹Mo-molybdate may be loaded onto a suitable column and kept in situ, eluting ^{99m}Tc-pertechnetate when required. Alternatively, after each elution the ⁹⁹Mo-molybdate could be eluted from the column and returned to the reservoir. The half-life of ⁹⁹Mo is, however, such that extended storage prior to disposal would be necessary to permit radioactive decay prior to disposal of single use columns. This and the more efficient use of the ⁹⁹Mo radioisotope, means that multiple use columns are preferred.

The method of the present invention may be carried out using laboratory robotics or an automated synthesizer. By the term “automated synthesizer” is meant an automated module based on the principle of unit operations as described by Satyamurthy et al [Clin. Positr. Imag., 2(5), 233-253 (1999)]. The term ‘unit operations’ means that complex processes are reduced to a series of simple operations or reactions, which can be applied to a range of materials. Such automated synthesizers are preferred for the method of the present invention, and are commercially available from a range of suppliers [Satyamurthy et al, above], including CTI Inc, GE Healthcare and Ion Beam Applications S.A. (Chemin du Cyclotron 3, B-1348 Louvain-La-Neuve, Belgium). Commercial automated synthesizers also designed to either provide suitable radiation shielding, or to be unshielded but located in a shielded hot cell (ie. a manufacturing cell specially designed for carrying out radiochemistry) to protect the operator from potential radiation dose. Such commercial synthesizers also comprise suitable containers for the liquid radioactive waste generated as a result of the radiopharmaceutical preparation.

Preferred automated synthesizers are those which comprise a disposable or single use cassette which comprises all the non-radioactive reagents, reaction vessels and apparatus necessary to carry out the preparation of a given batch of ^{99m}Tc radiopharmaceutical. Such cassettes are described in the second embodiment below. The cassette means that the automated synthesizer has the flexibility to be capable of making a variety of different ^{99m}Tc radiopharmaceuticals with minimal risk of cross-contamination, by simply changing the cassette.

It is envisaged that the process of the present invention can be used to produce a batch of a given ^{99m}Tc-labelled radiopharmaceutical which comprises sufficient radioactivity for almost any number of unit patient doses. The only constraint on the upper limit of doses is the volume of the reaction vessel and the radioactive concentration which can be achieved. The number of unit patient doses per batch is preferably 1 to 200, preferably 3 to 100, most preferably 5 to 50. The commercial automated synthesizer apparatus includes a detector for the automated measurement of the radioactive content and concentration of the reactants and products, so the radioactive content can be measured automatically. The batch can then be sub-dispensed into multiple unit doses suitable containers or clinical grade syringes as an additional feature of the present method, or the batch of several doses can be sub-dispensed as a separate exercise either manually or using a separate automated method, such as automated vial filling. The capability to produce multiple doses in this manner means that the present method is particularly useful in a radiopharmacy serving a patient population wherein many doses of the same ^{99m}Tc radiopharmaceutical are needed on the same day.

In a second aspect, the present invention provides a disposable cassette suitable for use in the method of the first embodiment, which comprises the reaction vessel and means for carrying out the transfer and mixing of step (iv) of the first embodiment, plus means for carrying out the manipulations of step (v) plus means for carrying out the optional additional process(es) of step (vi) of the method of the first embodiment.

By the term “cassette” is meant a piece of apparatus designed to fit removably and interchangeably onto an automated synthesizer apparatus (as defined above), in such a way that mechanical movement of moving parts of the synthesizer controls the operation of the cassette from outside the cassette, ie. externally. Suitable cassettes comprise a linear array of valves, each linked to a port where reagents or vials can be attached, by either needle puncture of an inverted septum-sealed vial, or by gas-tight, marrying joints. Each valve has a male-female joint which interfaces with a corresponding moving arm of the automated synthesizer. External rotation of the arm thus controls the opening or closing of the valve when the cassette is attached to the automated synthesizer. Additional moving parts of the automated synthesizer are designed to clip onto syringe plunger tips, and thus raise or depress syringe barrels.

The cassette is versatile, typically having several positions where reagents can be attached, and several suitable for attachment of syringe vials of reagents. The cassette always comprises a reaction vessel. Such reaction vessels are preferably 1 to 10 cm³, most preferably 2 to 5 cm³ in volume and are configured such that 3 or more ports of the cassette are connected thereto, to permit transfer of reagents or solvents from various ports on the cassette. Preferably the cassette has 15 to 40 valves in a linear array, most preferably 20 to 30, with 25 being especially preferred. The valves of the cassette are preferably identical, and most preferably are 3-way valves. The cassettes of the present invention are designed to be suitable for radiopharmaceutical manufacture and are therefore manufactured from materials which are of pharmaceutical grade and ideally also are resistant to radiolysis.

The cassettes comprise the various non-radioactive chemicals and reagents necessary for the preparation of a given ^{99m}Tc ligand metal complex. The cassettes are designed to be disposable, but also interchangeable. This means that, having invested in a relatively expensive automated synthesizer apparatus, the user can simply then purchase the cassettes as the consumables necessary. It is envisaged that a range of cassettes each having different ligands therein to generate different specific ^{99m}Tc radiopharmaceuticals would be used in conjunction with a given automated synthesizer apparatus.

The cassette preferably further comprises a supply of the reductant which may be in lyophilised, solution or solid phase form. Preferred aspects of the reductant are as described for the first embodiment above. The reductant of the cassette is preferably in solution in a biocompatible carrier medium, as defined above. A most preferred such solution is a sterile solution of stannous in a biocompatible carrier medium in the absence of air in a suitable container.

The cassette preferably further comprises a supply of the ligand. Preferred aspects of the ligand are as described for the first embodiment above. Most preferably, the ligand is supplied in kit form as described for the first embodiment above.

The cassette may optionally further include a supply of the radioactive materials necessary to prepare the desired ^{99m}Tc radiopharmaceutical, ie. either the ^{99m}Tc precursor solution or the ⁹⁹Mo-molybdate preferred aspect thereof, as described for the first embodiment above. When such radioactive materials are included with the cassette, appropriate radioactive shielding is envisaged also. It is preferred, however, that the cassette is non-radioactive.

The vials and containers of reagents of the cassette may optionally be colour-coded such that it is easier for the operator to identify the materials present. The various containers of the cassette may also optionally be identified distinctively in a computer-readable format (eg. bar code) to permit more facile microprocessor control and quality assurance. In a preferred embodiment, the whole cassette is identified distinctively in a computer-readable format (eg. bar code) so that the automated synthesizer can automatically check that the correct cassette is in place for the radiopharmaceutical to be prepared.

It is preferred that the cassette components, reductant and ligand are in sterile, apyrogenic form. Methods of sterilisation are as described above.

In a third aspect, the present invention provides the use of an automated synthesizer apparatus for the preparation of a ^{99m}Tc radiopharmaceutical. The “automated synthesizer” is as defined for the first embodiment above. Whilst such synthesizers have been used extensively for PET radiopharmaceuticals, their use for ^{99m}Tc is believed novel. This embodiment effectively relates to a novel method of using such automated synthesizers.

This automated synthesizer is preferably used to carry out by the method of the first embodiment, including preferred embodiments thereof.

The automated synthesizer used in this embodiment preferably comprises the disposable cassette of the second embodiment.

In a fourth aspect, the present invention provides the use of a sterile, non-radioactive kit for the preparation of a ^{99m}Tc radiopharmaceutical in the automated method of the first embodiment or the cassette of the second embodiment. This represents a new method of using conventional ^{99m}Tc kits.

Such kits, and preferred embodiments thereof are as described in the first embodiment above. In this fourth embodiment, the kits are designed to be reconstituted with a non-radioactive biocompatible carrier medium, as defined above, then the resulting solution is used in the automated method of the first embodiment. This is to be contrasted with the prior art, where kit reconstitution is with radioactive ^{99m}Tc-pertechnetate, followed by optional heating to give the radiopharmaceutical within the same vial or container.

In a fifth aspect, the present invention provides the use of the cassette of the second embodiment in the preparation of a ^{99m}Tc radiopharmaceutical. Preferably, the cassette is used in the process described in the first embodiment.

In a sixth aspect, the present invention provides a sterilised supply of ⁹⁹Mo-molybdate in a container suitable for pharmaceutical use. In this context, “sterilised” is as described above, and means that additional steps have been taken to sterilise the ⁹⁹Mo-molybdate to provide it in sterile, pyrogen-free form. Suitable methods of sterilisation are described above, and terminal sterilisation (eg. by sterile filtration, gamma-irradiation or autoclaving is preferred). Whilst highly-radioactive ⁹⁹Mo-molybdate is known in the prior art, the radioactivity alone cannot be assumed to give the degree of sterilisation necessary to remove pyrogens. In this embodiment, additional sterilisation steps are an essential feature.

The container suitable for pharmaceutical use, and preferred aspects thereof, is as described for the first embodiment above.

The sterilised supply of ⁹⁹Mo-molybdate is preferably provided in either aqueous solution or in solid form. Preferably the ⁹⁹Mo-molybdate aqueous solution is alkaline, most preferably dilute NaOH solution. One or more oxidising agent such as sodium hypochlorite solution may be added to the solution during processing, and such solutions are preferably stored under air, since that helps to maintain the high oxidation state of the molybdenum. Phosphate may optionally be added to produce phosphomolybdate solutions. The radiation dose of ⁹⁹Mo-molybdate means that suitable shielding must be used, preferably of tungsten or lead.

The invention is illustrated by the following non-limiting Examples. Example 1 demonstrates that a modified commercial automated synthesizer can be used successfully to prepare to prepare a known ^{99m}Tc radiopharmaceutical. Example 2 shows how the method of the present invention is useful to prepare ^{99m}Tc radiopharmaceuticals suitable for human administration which are not readily amenable to preparation via conventional kits. Example 3 shows how the method of the present invention can be used to remove excess non-radioactive ligand, which could potentially compete with the ^{99m}Tc radiopharmaceutical for the active target site in vivo.

### EXAMPLE 1

### Preparation of the Technetium Complex of TRODAT

The chelator TRODAT was prepared by the method of prepared in an analogous manner to Meegalla et al [J. Med. Chem., 40, 9-17 (1997)]. The lyophilised kit was prepared in an analogous manner to Kung et al [Nucl. Med. Biol., 26, 461-466 (1999)]. Decayed ^{99m}Tc generator eluate, ie. containing primarily ⁹⁹Tc-pertechnetate was used for this study.

A lyophilised kit for the preparation of ^{99m}Tc-TRODAT Injection containing:

| TRODAT-1 | 10 | μg* |
|---|---|---|
| SnCl₂•2H₂O | 38 | μg |
| Na-Glucoheptonate | 0 | |
| Na-Gluconate | 10 | mg |
| Na₂EDTA•2H₂O | 840 | μg |
| Na-Ascorbate | 500 | μg |

*Formulated as the trifluoroacetic acid salt

was fitted to a GE Healthcare Ltd FASTlab™ automated synthesizer. The following steps were then carried out automatically under control of the synthesizer software:
(i) decayed generator eluate (2.5 mL) was drawn from the reservoir into a syringe and from there injected into the kit;
(ii) the resulting solution was transferred to the reaction vessel where it was heated at 100° C. for 20 minutes;
(iii) the heated solution was then dispensed to a receiver vial and after cooling it was manually transferred to an autosampler vial for analysis by HPLC and pH testing.

### Results.

The chemical profile of the product was compared to an equivalent TRODAT kit vial reconstituted manually with decayed generator eluate (2.5 mL) using reverse-phase HPLC. The sample produced using the FASTlab™ gave peaks due to the two diastereomers of the Tc-complex at relative retention times of 19.5 and 21.0 minutes while the manually-reconstituted kit gave equivalent peaks at 20.6 and 21.7 minutes (measured on a separate machine). The pH of the FASTlab™ sample was 4.6 which is normal for reconstituted TRODAT kits.

### EXAMPLE 2

### Preparation of ^{99m}Tc(CO)₃-Containing Complexes

This is a prophetic Example of how such complexes could be prepared using the method of the present invention:

### Step (a): Preparation of [^{99m}Tc(CO)₃(H₂O)₃]⁺

This would be analogous to the literature method [Alberto et al, J. Am. Chem. Soc., 123, 3135-3136 (2001)], except that the method and cassette of the present invention would be employed. Thus, in a first step, pertechnetate would be introduced into an automated synthesizer apparatus, and would then be transferred to a reactor vessel where borax and boranocarbonate would be added. The reactor would be heated in the borate buffer at 95° C. for 20 minutes to produce [Tc(CO)₃(H₂O)₃]⁺. After cooling the solution would be neutralized with HCl and buffered with phosphate.

### Step (b): Preparation of ^{99m}Tc(CO)₃-Containing Ligand Complexes

The buffered solution of [Tc(CO)₃(H₂O)₃]⁺ from step (a) would then be heated with the ligand (optionally bound to a solid phase resin cartridge for ease of separation) for 30 minutes at 82° C., to form the desired ^{99m}Tc complex. Excess ligand would then be removed, by either solid phase binding as mentioned above or by use of HPLC or Sep-pak type cartridges), and the preparation analysed. Finally, the solution would be passed through clean-up cartridges to remove unreacted pertechnetate and toxic borate and reformulated as required ready for human injection.

### EXAMPLE 3

### Preparation of ^{99m}Tc Metal Complex of a Biologically Active Ligand Using a Kit Approach

This is a prophetic Example of how metal complexes of ligands which might potentially compete with the radiopharmaceutical for the biological site in vivo could be prepared using the method of the present invention and an automated synthesizer apparatus:

The following 10 step procedure would be used:

(i). water would be pumped into the freeze-dried kit;
(ii) the ligand would reconstituted non-radioactively in the vial;
(iii) ^{99m}Tc-pertechnetate solution would be transferred to the reactor of the automated synthesizer;
(iv) then ligand solution containing additives such as SnCl₂, buffers and optional stabilisers (eg. pABA) would be transferred to the reactor;
(v) radiolabelling would occur in the reactor with a chemical excess of ligand present over the technetium;
(vi) after completion of the radiolabelling the reaction mixture would be applied on eg. a reverse phase RP-18 Sep Pak SPE column;
(vii) salts and unreacted pertechnetate would be washed out from the SPE with water;
(viii) the desired ^{99m}Tc-ligand metal complex would be eluted from the column with ethanol, then solution transferred back to the reactor;
(ix) the ethanol would be evaporated under reduced pressure;
(x) the ^{99m}Tc-ligand metal complex would be reconstituted with an aqueous solution for formulation as a radiopharmaceutical.

In addition to the water reservoir 5 positions would be required on the automated synthesizer for this preparation:

(a) pertechnetate solution;
(b) freeze-dried kit;

### (c) EtOH;

(d) reverse phase column (SPE);
(e) reconstitution solution.

What is claimed is:

1. An automated method for the preparation of a sterile, ^{99m}Tc radiopharmaceutical composition which comprises a ^{99m}Tc metal complex in a biocompatible carrier medium, wherein said method comprises:
(i) provision of a precursor which comprises a solution of ^{99m}Tc-pertechnetate;
(ii) provision of a supply of a non-radioactive ligand, wherein said ligand forms a metal complex with ^{99m}Tc;
(iii) provision of a supply of a reductant capable of reducing technetium from the Tc(VII) oxidation state to a lower technetium oxidation state;
(iv) complexation of the ligand with ^{99m}Tc by microprocessor-controlled transfer of separate aliquots of said precursor and ligand to a reaction vessel and mixing therein, with optional heating, and optionally in the presence of an amount of said reductant effective to reduce said aliquot of ^{99m}Tc-pertechnetate precursor;
(v) when the ^{99m}Tc complex product from step (iv) is already in a biocompatible carrier medium it is used directly in step (vi), otherwise the product of step (iv) is either dissolved in a biocompatible carrier medium or the solvent used in step (iv) is removed and the residue re-dissolved in a biocompatible carrier medium;
(vi) optionally carrying out one or more of the following additional processes: purification; pH adjustment; solvent removal and re-dissolution in a biocompatible solvent to give the desired ^{99m}Tc radiopharmaceutical composition;
(vii) either maintaining sterility during steps (i) to (vi) so that the ^{99m}Tc metal complex from step (vi) is already sterile, or subjecting the ^{99m}Tc metal complex from step (vi) to either terminal sterilisation or sterile filtration to give the desired ^{99m}Tc-radiopharmaceutical.

2. The method of claim 1, where sterility is maintained during steps (i) to (vi) such that no additional terminal sterilisation step is necessary.

3. The method of claim 2, which further comprises providing the ligand in sterile solution by automated reconstitution with a suitable non-radioactive solvent of a kit containing the lyophilised ligand.

4. The method of claim 3, where the kit further comprises the reductant so that the ligand and reductant are provided as a lyophilised mixture.

5. The method of claim 4, where the lyophilised mixture of ligand and reductant is provided by a sterile, non-radioactive kit for the preparation of a ^{99m}Tc radiopharmaceutical.

6. The method of claim 5, where the ligand is chosen from: a phosphine, isonitrile, diaminedioxime, bis(aminothiol) or mercaptoacetyltriglycine (MAG3).

7. The method of claim 1, where an additional purification process step (vi) is included, which comprises the removal of unlabelled ligand to give a ^{99m}Tc radiopharmaceutical composition free of ligand.

8. The method of claim 1, where the reductant is biocompatible.

9. The method of claim 8, where the biocompatible reductant comprises stannous.

10. The method of claim 1, where the process is carried out using an automated synthesizer apparatus.

11. The method of claim 10, where the automated synthesizer apparatus comprises a disposable cassette which comprises the reaction vessel and means for carrying out the transfer and mixing of step (iv) plus means for carrying out the manipulations of step (v) and optional additional process(es) of step (vi).

12. The method of claim 1, which further comprises a reservoir sterile solution of ⁹⁹Mo-molybdate wherein the ^{99m}Tc-pertechnetate precursor of step (i) is provided by in situ radioactive decay of said ⁹⁹Mo to ^{99m}Tc, and said ^{99m}Tc-pertechnetate is separated from the ⁹⁹Mo-molybdate as part of the same automated process under microprocessor control.

13. A disposable cassette suitable for use in the method of claim 1, which comprises the reaction vessel and means for carrying out the transfer and mixing of step (iv) plus means for carrying out the manipulations of step (v) and means for carrying out the optional additional process(es) of step (vi).

14. The cassette of claim 13, which further comprises a supply of the reductant provision of a supply of a reductant capable of reducing technetium from the Tc(VII) oxidation state to a lower technetium oxidation state.

15. The cassette of claim 13, which further comprises a supply of the ligand wherein a supply of a non-radioactive ligand forms a metal complex with ^{99m}Tc.

16. The cassette of claim 15, where the ligand is supplied in a kit containing a lyophilised ligand.

17. The cassette of claim 13, which further comprises a supply of either ^{99m}Tc-pertechnetate precursor or ⁹⁹Mo-molybdate.

18. The cassette of claim 13, where the cassette components, reductant and ligand are in sterile, apyrogenic form.

19-25. (canceled)

26. A sterilised supply of ⁹⁹Mo-molybdate in a container suitable for pharmaceutical use.

27. The sterilised supply of claim 26, where the ⁹⁹Mo-molybdate is in a biocompatible carrier medium.

28. The sterilised supply of claim 26, where the ⁹⁹Mo-molybdate is in solid form.
